(19) United States
(12) Patent Application Publication
(10) Pub. No.: US 2007/0190102 A1
(43) Pub. Date: Aug. 16, 2007

(54) Method of preparing hydroxyapatite based drug delivery implant for infection and cancer treatment

(76) Inventor: Ping Luo, Emeryville, CA (US)

Correspondence Address: Berkeley Advanced Biomaterials, Inc., 2843 Forest Ave., Berkeley, CA, 94705, US

(21) Appl. No.: 10/913,689

(22) Filed: Aug. 9, 2004

Related U.S. Application Data
(63) Continuation-in-part of application No. 09/608,488, filed on Jun. 30, 2000, now Pat. No. 6,767,550.

(51) Int. Cl. A61F 2/02

(52) U.S. Cl. 424/423; 427/2.24

(57) ABSTRACT

A bioresorbable material is incorporated with bioactive agents to form an implant used for treatment against hard tissue or soft tissue defects and diseases. Antibiotics or anti-cancer agents are incorporated to treat hard or soft tissue infections or cancers. Sustained release of the bioactive agents or drug molecules may be achieved after implantation at the targeted sites. The dosage of the active agents or molecules, the microstructure, morphology, and composition of the bioresorbable material allow control of the release profile. The invented implant may be used for drug delivery, chemotherapy, or gene therapy. Various microstructure and the morphologies of the implants are injectable like putty or shaped with multilayers.

### TECHNICAL FIELD

This invention relates to the preparation of an implant containing anti-infection, anti-cancer, or anti-osteoporosis agents and pertains to the treatment of bone disease and soft tissue or breast cancers. The invented implants provide sustained release profiles after implantation. In this invention, we describe the composition of putty, spheres, granular/rod, and disc/tablet implants containing gentamycin, ciprofloxacin, doxorubicin, and other antibiotics, anticancer agents, and therapeutic agents. Homogenous and heterogeneous drug delivery implants with layered structures were described and prepared. Hydroxyapatite and composite biocompatible and bioresorbable materials are used to construct and fabricate the implants with layered structures.

### BACKGROUND OF THE INVENTION

Today, drugs are frequently administered orally in liquid or tablet forms. To treat cancer, cytotoxic drugs are used with the object of selective destruction of cancer cells. The major disadvantages of this therapy are their toxic effects on normal cells, and the rapid clearance of the drug from cancerous tissues [Kato, T., in Controlled Drug Delivery, Vol. 11, Clinical Applications, ed. Bruck, S.D., CRC Press, Boca Raton, FL, (1983) pp. 189-240]. To avoid problems incurred through the use of oral drugs, new dosage forms containing the drugs are introduced. There is a significant advantage to producing drug delivery systems that can maintain a constant drug release rate and can release the drug locally at the specific site of action. Therefore, implantable drug delivery systems were developed to optimize the therapeutic properties of the drug products and render them safer, more effective, and reliable. The advantages of drug delivery implants over conventional oral drugs are that:
1. a lower drug dose is needed,
2. the drug is protected from rapid in vivo metabolism,
3. the effectiveness of the drug at the site of the action is increased,
4. the patient compliance is increased and,
5. the delivery can continue over a period of time that can last for five years while requiring only minimum monitoring.
   Methods of Treating Bone Cancer, Bone Infection, and other Bone Diseases:

One of the important and effective drugs for treating osteosarcoma which is the most prevalent form of bone cancer is doxorubicin [Marsoni, S., Hoth, D., Simon, R., et al., Clinical Drug Development: An analysis of phase II trials, 1970-1985, Cancer Treat. Rep. 71, (1987) 71-80]. Since doxorubicin has poor oral absorption, it is administered intravenously. In the treatment of bone cancer, the problems associated with intravenous doxorubicin administration are: (i) toxicity of the drug; and, (ii) drug concentration at the cancerous site is likely to be very low because bones in general are moderately perfused organs. Administration of a 30 mg/m² of doxorubicin as an intravenous bolus dose resulted to a marro drug concentration of 0.52 μg/g, 2.5 hours after administration [Cohen, J. L., and Chan, K. K., in Bone Metastatsis Eds. Weiss, L. and Gilbert, H., A., Hall Medial Publishers, Boston, MA, (1981) pp. 276-299]. Cardiotoxicity is the major chronic toxicity of doxorubicin and is dose-dependent [Sadee, W. and Torti, F. M. , in Fundamentals of Cancer Chemotherapy, eds. Hellmann, K. and Carter, S. K., McGraw-Hill, New York, NY, (1987) pp. 19-27]. A cumulative dose of 700 mg.m causes 30-40% of the patients to experience cardiotoxicity.

The treatment of bone cancer in most cases involves surgical intervention followed by systemic chemotherapy. This therapy, commonly referred to as adjuvant chemotherapy, is used to eradicate microscopic foci of metastatic disease. Ettiger et al. used a combination of doxorubicin and cisplatin as adjuvant therapy to treat osteosarcoma patients. Eighty percent of their patients were continuously disease-free for 23 months [Ettiger, L. J., Douglas, H. O., Higby, D. J., et al., Adjuvant adriamycin and cis-diammine-dichloro-platinum in promary osteosarcoma, Cancer 47, (1981) 248-254]. Rosen et al. developed a very unconventional but successful treatment protocol which involved the following sequential steps: (i) a regimen of systemic chemotherapy initiated several weeks before surgery; (ii) resection of enoprosthetic replacement of tumor-bearing bone rather than amputation; (iii) histologic examination of resected primary tumor to evaluate the effect of the preoperative chemotherapy; and, (iv) initiation of a new postoperative chemotherapeutic regimen, if preoperative chemotherapy regimen was not effective [Rosen, G., Capparros, B., Huvos, A. G., et al., Preoperative chemotherapy for osteogenicsarcoma: selection of postoperative adjuvant chemotherapy based on the response of the primary tumor to preoperative chemotherapy, Cancer, 49(1982) 1221-1230].

This mode of treatment showed that 93% of the patients had been continuously disease free for 20 months. However, the systemic toxicity of doxorubicin was a cause for concern in some patients.

Hydroxyapatite based drug delivery implant can be used for treating bone infections and other soft tissue infections as well as other diseases such as osteoporosis. Hydroxyapatite based drug delivery implant can also treat osteoporosis effectively. Human bone is made of about 60% hydroxyapatite. Synthetic hydroxyapatite is resorbed and new bone is regenerated during resorbtion. After removing the cancer or tumor, the void can be filled with hydroxyapatite incorporated with anticancer drugs. Same voids caused by osteoporosis can be filled by hydroxyapatite drug livery implant. Bone regeneration proteins and peptides are often considered to be added into the implant to promote fast bone healing. For example, bone morphogenic proteins such as BMP2, BMP4, and BMP7 as well as commercially available growth factors, for example TGF and IGF can be incorporated into the hydroxyapatite based delivery implant. Other vitamins for example vitamin C, vitamin E, and vitamin D can also be added to enhance the treatment and assist effective delivery.

It has been known that bone morphogenetic protein (BMP) induces ectopic bone formation and plays an important role in the development of viscera. Ligand by binding to its receptor can induce a complex formation in which BMP2 receptor propagates the signal by phosphorylating a familty of signal transducers, the Smad proteins. There are 9 different Smad proteins. Upon phosphorylation by the BMP1 receptor, Smad1 can interact with either Smad4-Smad6 complex. The Smad1-Samd6 complex is inactive, but Smad1-Samd4 complex triggers the expression of BMP responsive genes. The ratio between Smad4 and Smad6 in the cell can modulate the strength of the signal transduced by BMP [Fujii, M. et al., Roles of bone morphogenetic protein type 1receptors and Smad proteins in osteoblast and chondroblast differentiation, Mol. Biol. Cell., 10 3801-3813 (1999) and Kawabata, M., et al., Signal transduction by bone morphogenetic proteins. Cytokine Growth Factor Rev., 9, 49-61 (1988)]. Transforming growth factor b-induced phosphorylation of Smad3 is required for growth inhibition and transcriptional induction in epithelial cells. Drosophila Mad proteins are intracellular signal transducers of decapentaplegic (dpp), the Drosophila transforming growth factor b (TGF-b)/bone morphogenic protein (BMP) homolog. In TGF-b treatment, Smad3 can be rapidly phosphorylated at the SSVS motif at its very C terminus. Phosphorylation of the three C-terminal serine residues of Smad3 by an activated TGF-b receptor complex is an essential step in signal transduction by TGF-b for both inhibition of cell proliferation and activation of the PAI-1 promoter. Smad3 plays an important role in the regulation of cell proliferation and transcriptional activation by the TGF-b receptors.

Insulinlike growth Factor I (IGF-I), a growth hormone-dependent peptide or somatomedin, plays also an important role on bone formation by examining the synthesis of DNA, collagen, and noncollagen protein. It is known that IGF-I increases the total collagen content of bones. The IGF-I stimulatory effect on the incorporation of [3H]thymidine was seen in the periosteum and periosteum-free calvarium. Not only IGF-I has effects on bone collagen synthesis but also IGF-I stimulates the synthesis of DNA at physiological concentrations [E. Canalis J Clin Invest. 1980 October; 66 (4): 709-719].

Biomolecules that enhance bone formation can be incorporated into drug delivery implant. Bone morphogenetic proteins (BMPs such as BMP1, BMP2, BMP3, BMP4, BMP5, BMP6, BMP7, BMP8, BMP9, and BMP10)and growth factors (for example

TGF-b and IGF-I promote fast bone healing after tumor is removal. Other small biomolecules including bone stimulating DNAs, peptides, amino acids (for example L-Arginine), enzymes, and hormones have been fund to be effective for promoting bone growth and wound healing in general. Combinations of various BMPs, growth factors, and small biomolecules may be very important to achieve fast bone healing.

Methods of treating Soft Tissue Sarcoma:

Soft tissue sarcomas are mesenchymal tumors arising from connective tissue elements grouped together based on a common biologic behavior. These tumors are relatively slow growing yet locally invasive with a high rate of recurrence following conservative management. Aggressive surgical resection, however, will often result in long term remission or cure. Chemotherapy for bulky disease has not been shown to be highly effective. Therefore, chemotherapy can not be considered a good option for initial therapy planning. Drugs that could be considered are doxorubicin, mitoxantrone and taxol for soft tissue sacroma. Treatment can be conducted by using intracavitary cisplatin released from a biodegradable polymer with preliminary local disease control. A porous biodegradable solid polymer termed Open cell PolyLactic Acid which is (OPLAa) impregnated with cisPlatin (OPLA-Pt) is placed within the wound following a marginal resection and prior to wound closure. This method results in cisplatin concentrations within the wound cavity which far exceed those obtainable with intravenous administration without high systemic concentrations which would result in toxicity. Such intracavitary therapy is effective treatment for microscopic disease.

Methods of treating Breast Cancer and other Cancers:

Doxorubicin is classified as an anthracycline antibiotic produced by Streptomyces peucetius. Doxorubicin, an antineoplastic, is found in two forms; free drug and methoxypolyethylene-glycol encapsulated liposomal form. The conventional form, Adriamycin, is used to treat a number of hematological malignancies and solid tumors including but not limited to Hodgkin's, sarcoma-osteogenic, leukemia, and breast, overies, lung, bladder and thyroid. Doxil, the liposomal form has become part of a standard treatment for AIDS-related Kaposi's sarcoma and third-line treatment of metastatic ovarian cancer.

In order to achieve effective delivery of doxorubicin or other protein drugs, peptides, and biomolecules, surgical implants are developed. Drug molecules can be imbedded into hydroxyapatite, calcium phosphates, and polymeric materials and surgically implanted into the body (affected area). The drug molecules are then released directly into the affected site via diffusion and surface resorbtion.

Biodegradable polymers are ones which degrade to smaller fragments by enzymes present in the body. They are: 1) natural polymers, which are always biodegradable; 2) modified nature polymers with various functional groups to enhance degradability; 3) chemical modification, in which the polymer structure is modified by reacting with highly reactive chemicals (for example crosslinking gelatin using formaldehyde and chitosan using glutaraldehyde); 4) enzymatic modification in a mild condition; and 5) synthetic polymers. Extensive reviews on the use of synthetic polymers in drug delivery are available in the literature (Langer, 1993; Heller, 1990; Peppas, 1991). Some of the polymers examined for use in drug delivery applications include polyanhydrides, polyesters, polyurethanes, polyphosphoesters, and polyphosphazenes. Hydrophilic polymers are more likely to be degradable than hydrophobic polymers. Polymers with heteroatoms in backbone is more degradable than polymers with C-C backbones. Amorphous polymer is more degradable than crystalline polymers. The higher the molecular weight, the lower is the degradability. Synthetic step-growth or condensation polymers are generally biodegradable to a certain extent.

### OBJECTIVE AND DISCLOSURE OF THE INVENTION

The objective of the present invention is to provide implants with layer structures that can deliver chemical or biological drugs, proteins, peptides, DNAs, amino acids, vitamins, enzymes, and hormones for treating infections and cancers. The designed implants in this invention can provide sustained release with tailored release profiles.

In this invention, anti infection and cancer drugs and active agents delivery implant composition and structures are disclosed. The compositions and methods can be also used to deliver agents such as therapeutics which have been plagued with delivery problems as well as traditional agents and can significantly reduce the effective dosages, increasing the therapeutic index and improving bioavailability thus reducing drug cytotoxicity and side effects.

In this invention different types of drugs and chemicals and biomolecules can be used alone or combined with other active or non-active agents to reach effective delivery. Conjugation of the biologic agent, such as active proteins and DNAs can be also delivered using parenteral implant in this invention. Conjugation of the biologic with albumin or other proteins encapsulated microbubbles can be also used for targeted delivery.

The term of “biomolecules” in this invention means chemical or biological drugs, proteins, amino acids, vitamins, peptides, DNAs, hormones, and cells.

In this invention, biomolecules are incorporated into hydroxyapatite based drug delivery implant to achieve sustained and tailored release profile. Combination of biomolecules are desired. In bone infection, cancer, and osteoporosis treatments, active drug molecules are incorporated into the implant to effective deliver the drug at local areas.

In this invention, for bone disease treatment, bone morphogenetic proteins and growth factors are incorporated into hydroxyapatite based implant to achieve sustained release and promote fast bone healing after tumor is removed. Possible proteins and peptides that can be incorporated into the delivery matrix include but not limited to TGF-b, IGF-I, L-Arginine, BMP1, BMP2, BMP3, BMP4, BMP5, BMP6, BMP7, BMP8, BMP9, and BMP10.

In this invention, for soft tissue treatment, biomolecules including soft tissue stimulating DNAs, peptides, amino, enzymes, and hormones, which have been fund to be effective for promoting fast wound healing, can be incorporated into the drug delivery implants after tumor removal.

In this invention, layered structures are described. Layered solid spheres, granular/rod or discs/tablets are to comprise two or more active substances to be released in a controlled release profile. The layers can two or more layers. The thickness of each layer can be from 1 angstrom to 10 mm. The layers can be either dense or porous matrices. This enables a release at desired rates. Active biomolecules diffuse through the matrix layers at different rates. In addition, active biomolecules that bind to the hydroxyapatite composite materials are released at different rates depending on the resorbtion rates of the matrix.

In this invention, the matrix layer contains hydroxyapatite based bioresorbable materials. Hydroxyapatite in this invention is either nanocrystals or microcrystals. The smaller the crystal size, faster the resorbtion rate thus the faster the release of bioactive agents and drug molecules. The inner layers contain fast resorbable compounds to build fast resorbable matrix. The outer layers contain slow resorbable compounds to build slow resorbable matrix. The faster resorbable compounds include gelatin, fibrin, collagen, biodegradable polymers such as PLLA, PLGA, PGA, magnesium oxide, calcium hydroxide, calcium oxide, calcium carbonate, tri-calcium phosphate, tetra-calcium phosphate octal-calcium phosphate, calcium sulfate calcium citrate, sodium and potassium silicates, nanocrystalline hydroxyapatite, calcium silicate. The slower resorbable compounds include glass, amorphous or crystalline silicon oxide, bioactive glass, aluminum silicate, zirconium silicate, microcrystalline hydroxyapatite, and slow or non-degradable polymers such as PMMA, polyethylenes, polyanhydrides, polyesters, polyurethanes, polyphosphoesters, and polyphosphazenes.

The layered implants can be processed on all standard machines to extrude, inject, mold or press and then processed by plasma coating, dip coating, so gel coating and further with finishing, etc.

The layered implants can be two layers or multilayers. They are made of combinations of various materials that have different resorbtion rates and structures to achieve tailored release characteristics. Under some circumstances, same materials are desired for all the layers with the same or various concentrations of biomolecules to achieve a sustained delivery profile.

As will become apparent, preferred features and characteristics of one aspect of the invention are applicable to any other aspects of the invention.

In one aspect, the invention provides a method for treating bone, cartilage and soft tissue infection and cancers including breast cancers using hydroxyapatite composite drug delivery implants.

In a preferred embodiment, the drug delivery implant includes either a single-phase hydroxyapatite or multi-phase calcium phosphates. In another preferred embodiment, the hydroxyapatite can be amorphous or crystalline. In another preferred embodiment, the phase of the calcium phosphate can be alpha-tri-calcium phosphate or beta-tri-calcium phosphate. In other preferred embodiments, the drug delivery implant is composed with at least one biocompatible material such as biocompatible polymer, collagen, bioactive glass, calcium sulfate, carbonate apatite, fluoroapatite, or a biocompatible apatite phase.

In another preferred embodiment, homogeneous or heterogeneous implants are prepared by controlling the composition of anti-cancer agents, the biocompatible materials and the pressing process. In another preferred embodiment, the pressure applied to form the granular, disc, tablet, or block implants ranges from 0.1 to 40 Mpa.

In another preferred embodiment, the invention includes using 0.02 weight percentage of doxorubicin to hydroxyapatite to obtain a sustained release. In another preferred embodiment, the drug molecules for treating infection or cancer various from 0.001% wt to 50% wt.

In another preferred embodiment, vitamins such as vitamin C, vitamin E, and vitamin D can be also incorporated into the delivery implant to assist the delivery.

In another preferred embodiment, the invention includes mixing doxorubicin and hydroxyapatite to form granular implants in a cylindrical rod shape. The diameter of the rod ranges from 5 microns to 10 millimeters.

In another preferred embodiment, the invention includes mixing doxorubicin and hydroxyapatite to form tablet or disc implants. The diameter of the tablet or disc ranges from 4 millimeters to 100 millimeters.

In another preferred embodiment, doxorubicin is placed in the inner layer matrix of hydroxyapatite and other slow resorbable compounds. Outer layer matrix is made by fast resorbable compounds without drug molecules, but with micro porous structure to allow slow release.

In another preferred embodiment, the invention essentially involves introducing granular and disc implants containing doxorubicin into the tumor or in its vicinity for treating bone or bone marrow, cartilage, soft tissues, or breast cancers.

In an embodiment, the implant consists of more than one layer, and that each of said layers is of a different material and/or comprises different active agents.

In another preferred embodiment, the following drug molecules and bioactive agents are incorporate alone or combined together into either inner layer matrices or outer layer matrices to achieve desired release profile.

In another preferred embodiment, the anti cancer drug agents, incorporated in the layered implant in this invention, are commercially available anticancer drugs and biomolecules that are chemically synthesized or marine or plant derived. They are either water soluble or insoluble. They are included but not limited to the following: aldesleukin, alemtuzumab, alitretinoin, allopurinol, altretamine, amifostine, amifostine, anastrozole, Ara-CMP, arsenic trioxide, asparaginase, BCG live, bexarotene, bleomycin, busulfan, calusterone, camptothecin sodium salt, capecitabine, carboplatin, carmustine with polifeprosan 20, celecoxib, chlorambucil, ciplatin, cladribine, cyclophosphamide, cytarabine, dacarbazine, dactinomycin, actinomycin, dacarbazin, darbepoetin, daunorubincin, denileukin diftitox, dexrazoxane, docetaxel, doxorubicin, dromostanolone propionate Elliott's B solution, epirubicin, epoetin, estramustine, etoposide phosphate, etoposide, exemestane, filgrastim, fluorouracil, floxuridine, fludarabine phosphate, fludarabine, fludarabine, fulvestrant, gemcitabine, gemtuzumab ozogamicin, goserelin acetate, hydroxyurea, ibritumomab tiuxetan, idarubicin, ifosfamide, imatinib mesylate, interferon alfa-2a or -2b, iphosphamide, irinotecan, letrozole, leucovorin, levamisole, Lomustine, mechlorethamine nitrogen mustard, megestrol acetate, hexamethyl and triethylene melamine, melphalan, L-PAM, mercaptopurine, 6-MP, mesna, methotrexate, metroxsalen, mithramycin, mitomycin C, mitotane, mitoxantrone, nandrolone phenpropionate, navelbine, nofetumomab, oprelvekin, paclitaxel, pamidronate, peqademase, pegfilgrastim, pentostatin, pipobroman, plicamydin, mithramycin, porfimer sodium, procarbazine, guinacrine, rasburicase, rituximab, sargramostim, streptozocin, talc, tamoxifen, temozolomide, teniposide, VM-26, testolactone, thioguanine, 6-TG, thiotepa, topotecan, toremifene, tositumomab, trastuzumab, trimetrexate, tretinoin ATRA, uracil mustard, valrubicin, vinblastine, vincristine, vinorelbine, and zoledronate. More other drugs are taxol/paclitaxel, vinblastine, vincristine, topotecan, iribinocan, etoposide, and teniposide.

In another preferred embodiment, anti-infection drugs incorporated in the hydroxyapatite layered implant in this invention, are commercially available antibiotics and other biomolecules that are chemically synthesized or marine or plant derived. Anti-infection drugs include but not limited to amoxicillan, ampicillin, augmentin, bactrim, blaxin, ceclor, ceftin, cephalexin, ciprofloxacin, clarinex, clindamycin, decadron, diclocil, difucan, doryx, doxyclycline, erythromyacin, flagyl, floxin, keflex, lincomycin, levoxil, macrobid, metrogel, metronizadole, minocin, neomycin, nizarol, norfloxacin, nystain, pnicillin, polarol, polymyxins, prednisone, rocefin, sporonox, sulfa, sulfameth/trimethroprin, taravid, tequin, tetnus, tetracycline, tinnidazole, tobramycin, valtrex, vancomycin, vibramcin, zamtac. Zithromax, zithromycin, zyrtec, zythromax.

In another preferred embodiment, anti-osteoporosis drugs such as prednisone or any steroid medications are incorporated into the drug delivery implant. One or more anti-osteoporosis drugs include but not limited to risedronate sodium, ibandronate sodium, etidronate disodium, raloxifene hcl, teriparatide, alendronate, and calcitonin.

### EXAMPLE 1

Gentamycin sulfate was dissolved into water and then mixed with hydroxyapatite and tricalcium powder into about 1.9% wt and 3% wt concentrations to make the inner layers of the implant. Spherical beads about 3 mm were made for testing. Outer layers of pure (without active agents) calcium sulfate faster resorbable compounds were then coated on the surface of the spherical beads about 0.5 mm by a dip coating method. The pore size is less than 500 microns. The formation of the spheres were conducted at room temperature. Processing temperature range can be selected according to the stabilities of the drug molecules (−4 to 150° C.). In vitro release testing was conducted to evaluate the release profile of this implant. Enclosed figure shows three release peaks. Gentamycin molecules diffuse out of the implant through the pores of the out layer of calcium sulfate at the beginning. Second peak demonstrated the release of gentamycin through dissolutions of calcium sulfate and tri-calcium phosphate. The third peak shows the last release of gentamycin from hydroxyapatite, which is a slower resorbable material compared with calcium sulfate and tri-calcium phosphate.

In the following figure, the histogram in the front represents the release profile from [1] 1.9% gentamycin sulfate (GS). The histogram in the back represents the release profile from [2] 3% gentamycin sulfate (GS).

[image]

### EXAMPLE 2

A powder mixture of hydroxyapatite, beta tricalcium phosphate, and calcium sulfate hemihydrate nanocrystalline powder matrix was used to bind bone morphogenetic proteins to fill bone voids due to osteoporosis. Bone morphogenetic proteins of BMP4 and BMP7 are purified up to 97% and freeze dried. A mixture of 50 to 50 ratio of BMP4 and BMP7 was then mixed with water and the powder matrix to make into injectable putty. The total protein content is about 0.5% wt. The putty is settable in 5 to 20 minutes in the bone void. In this case, proteins are bound more on to hydroxyapatite and tricalcium phosphate and less on to calcium sulfate in the powder matrix. Calcium sulfate works as a setting agent in this example. Bone morphogenetic proteins are the biomolecules that are sustained released out of the matrix.

1. A multiplayer, sustained release, biocompatible implant comprising inner layer consisting of slower resorbable hydroxyapatite composite materials with or without anti infection and cancer drug molecules and a outer layer comprising fast resorbable bioresorbable materials with or without drug molecules.

2. The implant in claim 1, wherein the inner layer further comprises multiphase materials selected from the group consisting of one or more calcium phosphates, nanocrystalline or microcystalline hydroxyapatite, calcium sulfate, calcium carbonate, calcium hydroxide, calcium oxide, bioactive glass, silica, silicon gels, silicates, aluminium oxides, biodegradable polymers (PLA and PGA), non-degradable PMMA, and resorbable collagen, fibrin and gelatins.

3. The implant in claim 1, wherein the outer layer further comprises multiphase materials selected from group consisting consisting of one or more calcium phosphates, nanocrystalline or microcystalline hydroxyapatite, calcium sulfate, calcium carbonate, calcium hydroxide, calcium oxide, bioactive glass, silica, silicon gels, silicates, aluminium oxides, biodegradable polymers (PLA and PGA), non-degradable PMMA, polyethylenes, polyanhydrides, polyesters, polyurethanes, polyphosphoesters, and polyphosphazenes, and resorbable collagen, fibrin and gelatins.

4. The drug molecules from claim 1, wherein the anticancer agents selected from the group consisting of one or more aldesleukin, alemtuzumab, alitretinoin, allopurinol, altretamine, amifostine, amifostine, anastrozole, Ara-CMP, arsenic trioxide, asparaginase, BCG live, bexarotene, bleomycin, busulfan, calusterone, camptothecin sodium salt, capecitabine, carboplatin, carmustine with polifeprosan 20, celecoxib, chlorambucil, ciplatin, cladribine, cyclophosphamide, cytarabine, dacarbazine, dactinomycin, actinomycin, dacarbazin, darbepoetin, daunorubincin, denileukin diftitox, dexrazoxane, docetaxel, doxorubicin, dromostanolone propionate Elliott's B solution, epirubicin, epoetin, estramustine, etoposide phosphate, etoposide, exemestane, filgrastim, fluorouracil, floxuridine, fludarabine phosphate, fludarabine, fludarabine, fulvestrant, gemcitabine, gemtuzumab ozogamicin, goserelin acetate, hydroxyurea, ibritumomab tiuxetan, idarubicin, ifosfamide, imatinib mesylate, interferon alfa-2a or -2b, iphosphamide, irinotecan, letrozole, leucovorin, levamisole, Lomustine, mechlorethamine nitrogen mustard, megestrol acetate, hexamethyl and triethylene melamine, melphalan, L-PAM, mercaptopurine, 6-MP, mesna, methotrexate, metroxsalen, mithramycin, mitomycin C, mitotane, mitoxantrone, nandrolone phenpropionate, navelbine, nofetumomab, oprelvekin, paclitaxel, pamidronate, peqademase, pegfilgrastim, pentostatin, pipobroman, plicamydin, mithramycin, porfimer sodium, procarbazine, guinacrine, rasburicase, rituximab, sargramostim, streptozocin, talc, tamoxifen, temozolomide, teniposide, VM-26, testolactone, thioguanine, 6-TG, thiotepa, topotecan, toremifene, tositumomab, trastuzumab, trimetrexate, tretinoin ATRA, uracil mustard, valrubicin, vinblastine, vincristine, vinorelbine, and zoledronate. More other drugs are taxol/paclitaxel, vinblastine, vincristine, topotecan, iribinocan, etoposide, and teniposide.

5. Multiplayer in claim 1 is two or more layers with or without porous structures.

6. Multilayer in claim 1 is either amorphous or crystalline.

7. Method of making inner layer in claim 1 comprises the step of:
a. Dissolving anti infection and cancer agents into water;
b. Mixing the solution of step (a) with powder, which is composed at least one of the bioresorbable materials to form a slurry or paste; and
c. Drying or molding the paste into spheres or granules/rods at a temperature ranging from −4 to about 150° C.

8. The method of making the inner layer drug delivery disk/tablet implant in claim 1 comprises the steps of:
a. Dissolving or without anti infection or cancer agents alone or with other chemicals or bioactive agents in water;
b. Mixing the solution of step (a) with a powder, which is composed at lease one of the bioresorbable materials to form a slurry or paste;
c. Drying the slurry from step (b) to form a solid at −4 to about 150° C.;
d. Crashing the solid from step c) into fine particles (this step can be eliminated of the drying processing in step (b) is controlled and results in fine particles); and
e. Pressing the particles from step d at a pressure ranging from 0.01 PSI to 3000 PSI to form a disk/tablet implant.

9. The method of making the outer layer in claim 1 comprises the step of
a. Dip coating the surface of tablet or granular implant with one or more hydroxyapatite composite and fast resorbable compounds selected from the group of calcium phosphates, calcium sulfates, aluminum silicate, sodium or potassium silicate, bioactive glass, gelatin, collagen, PLLA, and PGA and then following a drying process;
b. Or plasma spray coating the surface of the implant to form the outer layer;
c. Or chemical vapor or physical vapor deposition method to form the outer layer;
d. Or electrochemical deposition method to form the outer layer.

10. The implant in claim 1 is either an injectable putty or a solid material, wherein the structures and compositions of the inner layer and the outer layer are either same or different.

11. The implant in claim 1, wherein bone morphogenic proteins are incorporated to promote fast bone regeneration.

12. The implant in claim 1, wherein vitamins are imbedded.

13. The drug molecules in claim 1, where the concentration is from 0.001% to 50% wt.
